SEQUENCE LISTING (26 sequences)

SEQ ID NO: 1 - protein, 8 aa, Unknown
  Description of Unknown Protease cleavage site sequence
LEVLFQGP

SEQ ID NO: 2 - protein, 5 aa, Unknown
  Description of Unknown Protease cleavage site sequence
DDDDK

SEQ ID NO: 3 - protein, 4 aa, Unknown
  Description of Unknown Protease cleavage site sequence
IEGR

SEQ ID NO: 4 - protein, 7 aa, Unknown
  Description of Unknown Protease cleavage site sequence
ENLYFQG

SEQ ID NO: 5 - protein, 6 aa, Unknown
  Description of Unknown Protease cleavage site sequence
LVPRGS

SEQ ID NO: 6 - protein, 15 aa, Artificial Sequence
  Description of Artificial Sequence Synthetic peptide
GLNDIFEAQKIEWHE

SEQ ID NO: 7 - protein, 26 aa, Artificial Sequence
  Description of Artificial Sequence Synthetic peptide
KRRWKKNFIAVSAANRFKKISSSGAL

SEQ ID NO: 8 - protein, 6 aa, Artificial Sequence
  Description of Artificial Sequence Synthetic peptide
EEEEEE

SEQ ID NO: 9 - protein, 13 aa, Artificial Sequence
  Description of Artificial Sequence Synthetic peptide
GAPVPYPDPLEPR

SEQ ID NO: 10 - protein, 8 aa, Artificial Sequence
  Description of Artificial Sequence Synthetic peptide
DYKDDDDK

SEQ ID NO: 11 - protein, 9 aa, Artificial Sequence
  Description of Artificial Sequence Synthetic peptide
YPYDVPDYA

SEQ ID NO: 12 - protein, 6 aa, Artificial Sequence
  Description of Artificial Sequence Synthetic 6xHis
HHHHHH

SEQ ID NO: 13 - protein, 10 aa, Artificial Sequence
  Description of Artificial Sequence Synthetic peptide
EQKLISEEDL

SEQ ID NO: 14 - protein, 15 aa, Artificial Sequence
  Description of Artificial Sequence Synthetic peptide
KETAAAKFERQHMDS

SEQ ID NO: 15 - protein, 38 aa, Artificial Sequence
  Description of Artificial Sequence Synthetic polypeptide
MDEKTTGWRGGHVVEGLAGELEQLRARLEHHPQGQREP

SEQ ID NO: 16 - protein, 13 aa, Artificial Sequence
  Description of Artificial Sequence Synthetic peptide
SLAELLNAGLGGS

SEQ ID NO: 17 - protein, 8 aa, Artificial Sequence
  Description of Artificial Sequence Synthetic peptide
TQDPSRVG

SEQ ID NO: 18 - protein, 8 aa, Artificial Sequence
  Description of Artificial Sequence Synthetic peptide
WSHPQFEK

SEQ ID NO: 19 - protein, 6 aa, Artificial Sequence
  Description of Artificial Sequence Synthetic peptide
CCPGCC

SEQ ID NO: 20 - protein, 14 aa, Artificial Sequence
  Description of Artificial Sequence Synthetic peptide
GKPIPNPLLGLDST

SEQ ID NO: 21 - protein, 11 aa, Artificial Sequence
  Description of Artificial Sequence Synthetic peptide
YTDIEMNRLGK

SEQ ID NO: 22 - protein, 8 aa, Artificial Sequence
  Description of Artificial Sequence Synthetic peptide
DLYDDDDK

SEQ ID NO: 23 - protein, 16 aa, Artificial Sequence
  Description of Artificial Sequence Synthetic peptide
TDKDMTITFTNKKDAE

SEQ ID NO: 24 - protein, 13 aa, Artificial Sequence
  Description of Artificial Sequence Synthetic peptide
AHIVMVDAYKPTK

SEQ ID NO: 25 - protein, 489 aa, Artificial Sequence
  Description of Artificial Sequence Synthetic polypeptide
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGSGSGGGSSADGLKEPLSFHVIWIASFYNHSWKQNLVSGWLSDLQTHTWDSNSSTIVFLWPWSRGNFSNEEWKELETLFRIRTIRSFEGIRRYAHELQFEYPFEIQVTGGCELHSGKVSGSFLQLAYQGSDFVSFQNNSWLPYPVAGNMAKHFCKVLNQNQHENDITHNLLSDTCPRFILGLLDAGKAHLQRQVKPEAWLSHGPSPGPGHLQLVCHVSGFYPKPVWVMWMRGEQEQQGTQRGDILPSADGTWYLRATLEVAAGEAADLSCRVKHSSLEGQDIVLYWASWSHPQFEKASHHHHHHHHGGSLEVLFQGPGGSGGSDYKDDDDKEHHSSVGFIILAVIVPLLLLIGLALWFRKRCFC

SEQ ID NO: 26 - protein, 8 aa, Artificial Sequence
  Description of Artificial Sequence Synthetic 8xHis tag
HHHHHHHH

(19) United States
(12) Patent Application Publication
(10) Pub. No.: US 2019/0202890 A1
(43) Pub. Date: Jul. 4, 2019

(54) METHODS AND COMPOSITIONS RELATED TO ANTIGEN PRESENTING PROTEINS

(71) Applicant: THE REGENTS OF THE UNIVERSITY OF CALIFORNIA, Oakland, CA (US)

(72) Inventors: Kayvan R. Niazi, Agoura Hills, CA (US); Robert L. Modlin, Sherman Oaks, CA (US); Shahrooz Rabizadeh, Agoura Hills, CA (US)

(21) Appl. No.: 15/576,428

(22) PCT Filed: May 20, 2016
(86) PCT No.: PCT/US16/33474

§ 371 Date: Nov. 22, 2017

Related U.S. Application Data
(60) Provisional application No. 62/165,349, filed on May 22, 2015.

(51) Int. Cl. C07K 14/74; G01N 33/566; G01N 33/50

(52) U.S. Cl. CPC C07K 14/70539; G01N 33/566; G01N 33/5005; C07K 2319/50; C07K 2319/21; C07K 2319/22; C07K 2319/43; C07K 2319/03

(57) ABSTRACT

In some aspects, the invention relates to a transmembrane protein comprising an extracellular domain that may be cleaved from a transmembrane domain by a protease. The extracellular domain may be the extracellular domain of an antigen-presenting protein, such as CD1a.

### RELATED APPLICATIONS

This patent application claims priority to U.S. Provisional Patent Application No. 62/165349, filed May 22, 2015, which is hereby incorporated by reference in its entirety.

### GOVERNMENT INTEREST

This invention was made with Government support under AR40312, awarded by the National Institutes of Health. The Government has certain rights in the invention.

### BACKGROUND

The human CD1 proteins are major histocompatibility complex-(MHC)-related proteins which present lipoglycan/glycolipid antigens to T cells. The human CD1 family consists of five isoforms (CD1a-e), which have evolved unique structural and intracellular trafficking features that enable them to present different classes of hydrophobic antigens collected from various endocytic compartments (e.g., endosomes, phagosomes, lysosomes). Until now, the majority of knowledge regarding CD¹/antigen interactions has relied either on: 1) the artificial loading of purified soluble recombinant proteins in cell-free systems, 2) cell culture systems utilizing a previously identified single antigen or groups of related structures, or 3) some combination of the above. These approaches severely restrict CD1 ligand identification by their intrinsic operator-based biases such as the use of individual antigens derived from a milieu, which are likely to contain many other molecules.

### BRIEF DESCRIPTION

In some aspects, the invention relates to a transmembrane protein comprising an extracellular domain that may be cleaved from a transmembrane domain by a protease. The extracellular domain may be the extracellular domain of an antigen-presenting protein, such as CD1a. Some aspects of the invention relate to nucleic acids encoding a protein described herein or cells comprising said nucleic acids. Other aspects of the invention relate to methods of purifying and/or isolating proteins and/or antigens.

### DESCRIPTION OF THE FIGURES

FIG. 1 shows the design and amino acid sequence (SEQ ID NO:25) of a protein comprising a β-2m extracellular domain (β2M), glycine-serine linker (GS), CD1a extracellular domain (CD1a EC), streptavidin affinity tag (Strep), histidine affinity tag (8 His), 3C protease cleavage site (Cleave), FLAG affinity tag (Flag), transmembrane domain (TM), and cytosolic domain (CT).

FIG. 2 shows flow cytometry results for proteins comprising a β-2m extracellular domain, CD1a extracellular domain, and 3C protease cleavage site expressed in mouse B16 cells, before and after cleavage with 3C protease, using fluorescence labels for CD1a and β-2m. “Mock” depicts control cells that were not transfected, “cCD1al” and “cCD1a2” depict cells that were transfected with a cleavable protein comprising the β-2m and CD1a extracellular domains, and “WT CD1a” depicts cells that were transfected with wild type human CD1a, which lacks a β-2m extracellular domain and 3C protease cleavage sequence.

FIG. 3 consists of two panels, labeled panel (A) and panel (B). Panel (A) shows flow cytometry results for proteins comprising a CD1a extracellular domain and 3C protease cleavage site expressed in eukaryotic cells, before and after cleavage with 3C protease (CD1a and CD1a+3C, respectively). Panel (B) shows a polyacrylamide gel of protein cleaved from cells with a 3C protease and purified using Ni-NTA, to which the histidine tag binds. The “elution” lanes correspond to protein purified using Ni-NTA, and the molecular weights of the major bands correspond to the molecular weight of the cleaved extracellular domain.

FIG. 4 consists of three panels, labeled (A), (B), and (C). Molecules bound to cCD1a were eluted from Ni-NTA resin using N-propanol (A), chloroform-methanol-water (B; 10:10:3), and chloroform-methanol (C; 2:1), and their molecular weights were measured using mass spectroscopy. White diamonds correspond to molecules eluted from cCD1a after incubating the cCD1a-expressing cells with a lysate from M. tuberculosis. Black diamonds correspond to a control, for untreated cCD1a-expressing cells.

FIG. 5 is a graph showing interferon γ (INF-γ) concentration (y-axis) induced by various concentrations of lipoprotein LppX (LppX; x-axis). HeLa cells transfected with wild type CD1a (wtCD1 A), an engineered protein comprising a cleavable CD extracellular domain (cCDA1), and untransfected HeLa cells were incubated with LppX and LCD4.G T cells, which are a CD1A-restricted T cell clone that recognize a LppX antigen. INF-γ production by the T cells was measured by ELISA. cCD1A-transfected cells displayed a similar ability to induce INF-γ production as wtCD1A-transfected cells, suggesting that the cCD1A construct maintained its ability to present antigen to T cells.

### DETAILED DESCRIPTION

In some aspects, the invention relates to a protein comprising an extracellular domain from a CD1 glycoprotein. The protein may comprise a cytosolic domain from a CD1 glycoprotein. Previous studies have described a variety of protein domains with the ability to differentially distribute genetically fused protein partners to distinct intra- and extra-cellular locations, often times with dramatic differences in resulting function. More specifically, expression cassettes comprising sequences borrowed from the human CD1 proteins may be used to traffic genetically-fused heterologous proteins to different intracellular locations (see, e.g., Niazi, K. R. et al., Immunology 122(4):522-31 (2007)). Like most type 1 membrane proteins, the CD1 genes encode polypeptides with unique subdomains, each with an associated function. In the case of CD1, there are 4 domains, with the N-terminus of the protein (i.e., “domain 1”) encoding a leader peptide, a sequence which targets the CD1 messenger RNA and associated ribosome to the endoplasmic reticulum for translocation of the remaining protein into its lumen. In essence, the synthesis of the leader peptide is the first step in targeting a type 1 membrane protein for the secretory system and the extracellular space rather than the cytoplasm. After the leader peptide sequence, the second CD1 subdomain (“domain 2”) is an extracellular domain with sequence and structural homology to MEC I, which binds lipoglycan antigens for presentation to T lymphocytes. To anchor this extracellular domain to the cell membrane, the next module in the wild type CD1 sequence (“domain 3”) is a transmembrane domain, a stretch of 15 or more hydrophobic or apolar amino acid residues that span the width of the plasma membrane. The final domain of CD1 (“domain 4”) is a cytoplasmic tail sequence which serves as a capture sequence by intracellular adaptor proteins for trafficking CD1 from the membrane to distinct intracellular endosomal vesicles. Using each of these domains as independent functional units, the extracellular domains of the CD1 proteins may be replaced with fusion proteins, such as GFP, mycobacterial GroES, and others, to target them to the compartments to which the wild type CD1s would traffic, thereby creating a panel of targeting cassettes (see, e.g., Niazi, K. R. et al., Immunology 122(4):522-31 (2007)).

Some aspects of the instant invention include additional novel features, such as an extracellular, membrane proximal proteolytic cleavage site, e.g., comprising the recognition sequence of the picornavirus 3C protease, and a purification domain, e.g., comprising one or more “affinity tags”, such as a stretch of six to eight histidines (8 His) or a Strep-Tag sequence. The 3C recognition site may be selected in part due to the ability of 3C protease to cleave its substrate at physiological pH and salt concentrations, thereby preventing cellular lysis and subsequent contamination of the target protein pool by intracellular proteins. The location of the affinity tags may vary between N-terminal, C-terminal, or internal, depending on the degree of functional tolerance of the protein partner, and the cleavage sequence is ideally located closer to the membrane for type 1 membrane proteins. These modules , may also be used in combination with type 2 integral membrane proteins.

### I. Recombinant Proteins

In some aspects, the invention relates to a protein comprising an extracellular domain, a transmembrane domain, and a protease cleavage site located between the extracellular domain and the transmembrane domain. The extracellular domain may comprise the extracellular domain of an antigen-presenting protein. The protease cleavage site may be located in proximity to the transmembrane domain. For example, the protease cleavage site may be located within 50, 40, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1amino acids from the transmembrane domain.

The protease cleavage site may be recognized by a protease that recognizes at least 4 amino acids, such as a protease that recognizes at least 5, 6, 7, or 8 amino acids. The protease cleavage site may be 4, 5, 6, 7, 8, 9, or 10 amino acids, i.e., the protease cleavage site may be recognized by a protease that recognizes a sequence of 4, 5, 6, 7, 8, 9, or 10 amino acids. The protease cleavage site may be recognized, for example, by thrombin, factor Xa, TEV protease, enteropeptidase, or rhinovirus 3C protease, i.e., the protease cleavage site may be a thrombin, factor Xa, TEV protease, enteropeptidase, or rhinovirus 3C protease cleavage site. In some embodiments, the protease cleavage site is recognized by 3C protease, i.e., the protease cleavage site may be a 3C protease cleavage site. The nature of the protease cleavage site is not particularly limiting, however, so long as the protease is specific. The protease cleavage site may be, for example, LEVLFQGP (SEQ ID NO:1; cleaved by rhinovirus 3C protease); DDDDK (SEQ ID NO:2; cleaved by enteropeptidase); IEGR (SEQ ID NO:3; cleaved by Factor Xa); ENLYFQG (SEQ ID NO:4; cleaved by TEV protease); or LVPRGS (SEQ ID NO:5; cleaved by thrombin protease). In some embodiments, the protease cleavage site comprises SEQ ID NO:1.

The extracellular domain may comprise the extracellular domain of a CD1 protein, such as the extracellular domain of CD1a, CD1b, CD1c, CD1d, or CD1e. In some embodiments, the extracellular domain comprises the extracellular domain of CD1a. The CD1 protein may be human CD1. In some embodiments, the extracellular domain comprises the extracellular domain from β-2 microglobulin (β-2M). In some embodiments, the extracellular domain comprises the extracellular domain from a MEC class I alpha chain.

The extracellular domain may comprise a portion of the extracellular domain of a CD1 protein, such as a portion of the extracellular domain of CD1a, CD1b, CD1c, CD1d, or CD1e. In some embodiments, the extracellular domain comprises a portion of the extracellular domain of CD1a. The CD1 protein may be human CD1. In some embodiments, the extracellular domain comprises a portion of the extracellular domain from β-2M. In some embodiments, the extracellular domain comprises a portion of the extracellular domain from a MEC class I alpha chain. In some embodiments, the extracellular domain comprises the antigen-presenting domain of a protein.

In some embodiments, the transmembrane domain is a single alpha helix. The protein may be a type 1 membrane protein, i.e., the protein may be oriented such that the extracellular domain is the N-terminus of the protein and the cytosolic domain is the C-terminus.

In some embodiments, the protein further comprises a first affinity tag located between the N-terminus of the protein and the protease cleavage site. The first affinity tag may be located between the extracellular domain and the protease cleavage site.

In some embodiments, the protein further comprises a second affinity tag located between the C-terminus of the protein and the protease cleavage site. The second affinity tag may be located between the transmembrane domain and the protease cleavage site.

The nature of the first affinity tag and second affinity tag is not particularly limiting. For example, at least one of the first affinity tag and second affinity tag may be selected from AviTag (SEQ ID NO:6 GLNDIFEAQKIEWHE), Calmodulin-tag (SEQ ID NO:7 KRRWKKNFIAVSAANRFKKISSSGAL), polyglutamate tag (SEQ ID NO:8 EEEEEE), E-tag (SEQ ID NO:9 GAPVPYPDPLEPR), FLAG-tag (SEQ ID NO:10 DYKDDDDK), HA-tag (SEQ ID NO:11 YPYDVPDYA), His-tag (SEQ ID NO:12 HHHHHH), Myo-tag (SEQ ID NO:13 EQKLISEEDL), S-tag (SEQ ID NO:14 KETAAAKFERQHMDS), SBP-tag (SEQ ID NO:15 MDEKTTGWRGGHVVEGLAGELEQLRARLEHHPQGQREP), Softag 1 (SEQ ID NO:16 SLAELLNAGLGGS), Softag 3 (SEQ ID NO:17 TQDPSRVG), Strep-tag (SEQ ID NO:18 WSHPQFEK), TC tag (SEQ ID NO:19 CCPGCC), V5 tag (SEQ ID NO:20 GKPIPNPLLGLDST), VSV-tag (SEQ ID NO:21 YTDIEMNRLGK), Xpress tag (SEQ ID NO:22 DLYDDDDK), Isopeptag (SEQ ID NO:23 TDKDMTITFTNKKDAE), and SpyTag (SEQ ID NO:24 AHIVIVIVDAYKPTK). In some embodiments, the first affinity tag comprises strep-tag (SEQ ID NO:18) and/or his-tag (SEQ ID NO:12). In some embodiments, the second affinity tag comprises FLAG-tag (SEQ ID NO:10).

In some embodiments, the protein comprises a N-terminal leader sequence, e.g., for translocating the extracellular domain across a membrane. In some embodiments, the protein comprises a cytosolic domain, e.g., for trafficking the protein within a cell. The cytosolic domain may comprise the cytosolic domain of a CD1 protein, such as CD1a, CD1b, CD1c, CD1d, or CD1e. For example, the cytosolic domain may comprise a portion of the human CD1a cytosolic domain.

The protein may comprise at least about 95%, 96%, 97%, 98%, or 99% sequence identity with the amino acid sequence set forth in SEQ ID NO:25. The protein may have the amino acid sequence set forth in SEQ ID NO:25.

### II. Recombinant Nucleic Acids

In some aspects, the invention relates to a nucleic acid encoding any one of the proteins described herein. The nucleic acid sequence that encodes the protein is referred to as a “gene” herein. The nucleic acid may further comprise a promoter operably linked to a nucleotide sequence encoding any one of the proteins described herein. The nucleic acid may further comprise a selectable marker, such as an antibiotic resistance gene. The nucleic acid may further comprise an origin of replication, e.g., for cloning the nucleic acid in a cell, such as E. coli.

### III. Cells

In some aspects, the invention relates to a cell comprising a gene (i.e., a recombinant gene) encoding any one of the proteins described herein. The cell may be a prokaryotic cell, e.g., for cloning the gene. The cell may be a eukaryotic cell, e.g., for expressing the protein. The gene may be present on a plasmid. In some embodiments, the gene is not present on a plasmid, e.g., after stable transfection of an expression cell. The gene may be integrated into the genome of a cell, i.e., after transformation or transfection of a cell with a nucleic acid encoding the gene. The cell may comprise a nucleic acid comprising a gene encoding the protein and a selectable marker, e.g., a selectable marker associated with the gene, for selecting cells that comprise the gene. The cell may be a eukaryotic cell, and the gene may be integrated into the genome of the cell.

In some embodiments, the cell is a cloning cell, e.g., the cell may be selected from E. coli and S. cerevisiae.

The cell may be selected from C6/36, S2, Sf21, Sf9, and High Five cells.

In some embodiments, the cell is a eukaryotic cell and the cell expresses the protein. The cell may be a mammalian cell, such as a mouse cell or a human cell. In certain preferred embodiments, the cell is a human cell. The cell may be selected from 721, 293T, A172, A253, A2780, A2780ADR, A2780cis, A431, A-549, BCP-1 , BEAS-2B, BR 293, BT-20, BxPC3, Cal-27, CML T1, COR-L23, COR-L23/5010, COR-L23/CPR, COR-L23/R23, COV-434, DU145, DuCaP, EM2, EM3, FM3, H1299, H69, HCA2, HEK-293, HeLa, HMEpC, HT-29, HUVEC, Jurkat, JY, K562, KBM-7, KCL22, KG1, Ku812, KYO1, LNCap, Ma-Mel , MCF-10A, MCF-7, MDA-MB-157, MDA-MB-231, MDA-MB-361, MG63, MONO-MAC 6, MOR/0.2R, MRCS, NCI-H69/CPR, NCI-H69/LX10, NCI-H69/LX20, NCI-H69/LX4, Peer, Raji, Saos-2, SiHa, SKBR3, SKOV-3, T2, T-47D, T84, U373, U87, U937, VCaP, WM39, WT-49, and YAR cells. The cell may be selected from 3T3, 4T1, A20, ALC, B16, bEnd.3, C2C12, C3H-10T1/2, CGR8, CT26, E14Tg2a, EL4, EMT6/AR1, EMT6/AR10.0, Hepalclc7, J558L, MC-38, MTD-1A, MyEnd, NIH-3T3, RenCa, RIN-5F, RMA/RMAS, X63, and YAC-1 cells. The cell may be selected from 9L, B35, MK-21, C6, CHO, CMT, COS-7, D17, DH82, MDCK II, RBL, and Vero cells. The cell may be the cell of an immortalized cell line, a peripheral blood mononuclear cell, or a fibroblast.

In some embodiments, the cell is from an organism that does not express a protease that can cleave the protein at the protease cleavage site. In preferred embodiments, the cell is from an organism that expresses the antigen-presenting protein. In some embodiments, the protein comprises an N-terminal leader sequence for translocating the extracellular domain across a membrane of the cell; and the cell is from an organism that expresses the N-terminal leader sequence on a native protein. In some embodiments, the protein comprises a cytosolic domain for trafficking the protein within the cell; and the cell is from an organism that expresses the cytosolic domain.

The cell may or may not endogenously express a CD1 protein. “Endogenous expression” as described herein refers to the expression of a protein irrespective of transfection with a gene described herein, i.e., wherein the protein is expressed from mRNA that is transcribed from a native gene in the cell rather than a gene that is introduced by transfection. The cell may or may not endogenously express CD1a, CD1b, CD1c, CD1d, and/or CD1e. In certain embodiments, the cell does not endogenously express CD1 (e.g., CD1a, CD1b, CD1c, CD1d, and/or CD1e), and the cell expresses a protein comprising the extracellular domain of the antigen-presenting protein CD1a as described herein.

The cell may or may not endogenously express a class I major histocompatibility complex (MHC Class I). The cell may or may not endogenously express a class II major histocompatibility complex (WIC Class II). The cell may or may not be an antigen-presenting cell.

### IV. Methods for Isolating an Antigen

In some aspects, the invention relates to a method for isolating an antigen. The method may comprise contacting a cell as described herein, supra, with a mixture comprising the antigen; incubating the cell with a protease that recognizes the protease cleavage sequence of a protein (as described herein) expressed by the cell, thereby cleaving the extracellular domain of the protein from the cell; and isolating the extracellular domain of the protein from the mixture, thereby isolating the antigen bound to the extracellular domain. In some embodiments, the method comprises contacting a cell as described herein, supra, with a mixture comprising the antigen; isolating the cell from the mixture; incubating the cell with a protease that recognizes the protease cleavage sequence of a protein (as described herein) expressed by the cell, thereby cleaving the extracellular domain of the protein from the cell; and isolating the extracellular domain from the cell, thereby isolating the antigen. In the foregoing methods, the cell is preferably a eukaryotic cell, more preferably a mammalian cell, such as a mouse cell or a human cell. The protein may comprise an affinity tag. Isolating the cell from the mixture may or may not comprise incubating the mixture with a molecule that specifically binds the affinity tag. Isolating the cell may comprise pelleting the cell and removing the supernatant, fluorescence-activated cell sorting (FACS), or magnetic-activated cell sorting (MACS). The protein may comprise an affinity tag between the extracellular domain and protease cleavage site. Thus, isolating the extracellular domain may comprise incubating a composition comprising the extracellular domain (e.g., the mixture) with a molecule that specifically binds the affinity tag. Isolating the extracellular domain may comprise affinity chromatography, e.g., with a stationary phase that specifically binds the affinity tag. The method may further comprise isolating the antigen from the extracellular domain. For example, the method may comprise contacting a complex comprising the antigen, the extracellular domain, and a molecule that specifically binds the affinity tag with a chemical denaturant, such as urea or guanidine, while the extracellular domain is bound to the molecule that specifically binds the affinity tag, and separating the antigen from the complex (e.g., by affinity chromatography, centrifugation, filtering, or magnetic separation). Alternatively, isolating the antigen from the extracellular domain may comprise a chromatography, such as high performance liquid chromatography (HPLC). The method may further comprise identifying the antigen. For example, the method may comprise mass spectroscopy (e.g., HPLC-MS, LC-MS, single quadrupole, triple quadrupole, ion trap, time-of flight, quadrupole-time of flight, and/or tandem MS).

A molecule that specifically binds the affinity tag may be attached to a particle, bead, resin, or other solid support structure (e.g., covalently attached). The particle, bead, resin, or other solid support structure may allow for purification by centrifugation, filtering, affinity chromatography, or magnetic separation. The molecule may be attached to a fluorophore (e.g., covalently attached).

### V. Methods for Isolating a T Cell

In some aspects, the invention relates to a method for isolating a T cell, comprising contacting a cell (e.g., that expresses a protein as described herein) with an antigen; incubating the cell with a plurality of T cells; isolating a T cell that binds to the cell, thereby isolating the T cell; and incubating the cell with a protease that recognizes the protease cleavage site of the protein, thereby cleaving the T cell from the cell. At least some of the T cells of the plurality are preferably capable of specifically binding to an antigen/antigen-presenting protein complex such that some T cells of the plurality could specifically bind to the extracellular domain of the protein expressed on the cell if the extracellular domain presented an antigen recognized by the T cell. Thus, the extracellular domain of the protein expressed on the cell is preferably of the same species as the T cells of the plurality. The cell is preferably of the same species as the T cells of the plurality.

Contacting the cell with the antigen may comprise either adding the antigen to a composition comprising the cell, thereby resulting in a composition comprising the cell and the antigen; or adding the cell to a mixture comprising the antigen, thereby resulting in a composition comprising the cell and the antigen. The mixture comprising the antigen may comprise the plurality of T cells. Alternatively, the composition comprising the cell may comprise the plurality of T cells. Alternatively, the method may comprise adding the plurality of T cells to s composition comprising the cell and the antigen.

The plurality of T cells may comprise (or consist essentially of) CD1-restricted T cells, e.g., when the protein comprises the extracellular domain of a CD1 family member, such as CD1a. The plurality of T cells may comprise (or consist essentially of) CD1a, CD1b, CD1c, and/or CD1d-restricted T cells, e.g., when the protein comprises the extracellular domain of a corresponding CD1 family member.

The method may further comprise isolating the T cell from the cell.

Isolating the T cell that binds to the cell and/or isolating the T cell from the cell may comprise fluorescence-activated cell sorting (FACS) or magnetic-activated cell sorting (MACS). For example, the method may comprise contacting the cell with a molecule that specifically binds an affinity tag of the protein expressed by the cell, e.g., wherein the molecule is attached to a fluorophore or a magnetic, paramagnetic, or superparamagnetic particle, thereby allowing isolation of the T cell either bound to the cell (i.e., prior to incubating with the protease) or isolation of the T cell from the cell (i.e., after incubating with the protease).

After the T cell is isolated from other T cells of the plurality and the cell, then the T cell may be expanded and/or characterized. For example, the method may comprise sequencing the complementarity determining regions of a T cell receptor of the T cell, e.g., to identify amino acid sequences that specifically bind the antigen. The method may comprise sequencing the complementarity determining regions of an αβ T cell receptor (αβ TCR) and/or a γδ T cell receptor (γδ TCR), e.g., wherein the protein comprises the extracellular domain of a CD1 family member.

This disclosure will be better understood from the Experimental Details which follow. However, one skilled in the art will readily appreciate that the specific methods and results discussed are merely illustrative of the disclosure as described more fully in the embodiments which follow thereafter.

### Exemplification

### EXAMPLE 1

### Design of a Cleavable Extracellular Domain

A CD1a/β-2 microglobulin (β-2m) fusion protein was used as a model protein. Since β-2m possesses its own leader peptide, this domain was not replaced with the functionally equivalent sequence from CD1a, but any leader peptide or signal sequence may be used so long as it targets the protein to the membrane of the expression cell. The construct takes advantage of the ability of CD1a to bind lipoglycan, to provide functional evidence of the utility of the surface expression, cleavage, and purification system under development. As a result, CD1a systems provide a direct means of sampling the pool of authentically-loaded CD1a-bound antigens following their uptake, processing, and presentation by live cells, thereby removing operator biases. To create a recombinant CD1a protein capable of intracellular traffic, on-demand cleavage from the cell surface, and easy purification, the following construct was designed.

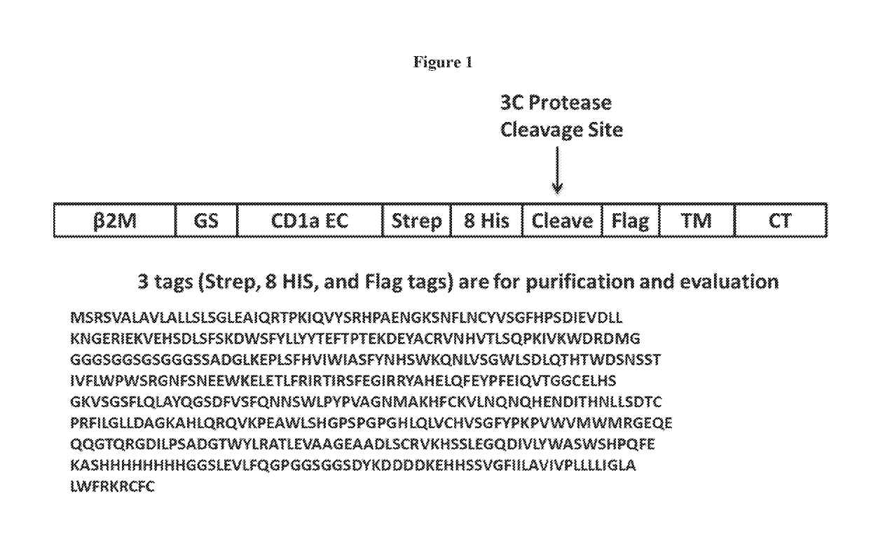

A chimeric gene was cloned encoding human β-2 microglobulin (β2M), a glycine-serine linker (GS), an mature CD la extracellular domain (CD1a EC), a Strep-tag sequence (Strep), a poly-histidine affinity tag (8 His), a human rhinovirus 3C protease motif (Cleave), a glycine-serine linker and FLAG epitope (FLAG), and the wild-type CD1a transmembrane and cytoplasmic domains (TM and CT, respectively) using a cassette-based cloning scheme utilizing overlapping oligonucleotide-based polymerase chain reaction (see FIG. 1). The resultant expressed protein is not secreted, but traffics in a manner similar to wild-type CD1a, enabling cleavage and purification following cellular antigen processing and loading.

The sequence encoding the β-2m leader peptide served as domain 1, the remaining β-2m, glycine-serine linker, and extracellular domain of CD1a represent domain 2, the purification and cleavage module were inserted between domains 2 and 3, the FLAG-tagged CD1a transmembrane domain serves as domain 3, and the cytoplasmic domain of CD1a represents domain 4. The CD1a cytoplasmic tail, though not unique in its ability, is an ideal targeting sequence because it can traffic fused protein partners predominantly to the cell surface or recycling endosomes.

### EXAMPLE 2

### Expression and Cleavage of the Fusion Protein

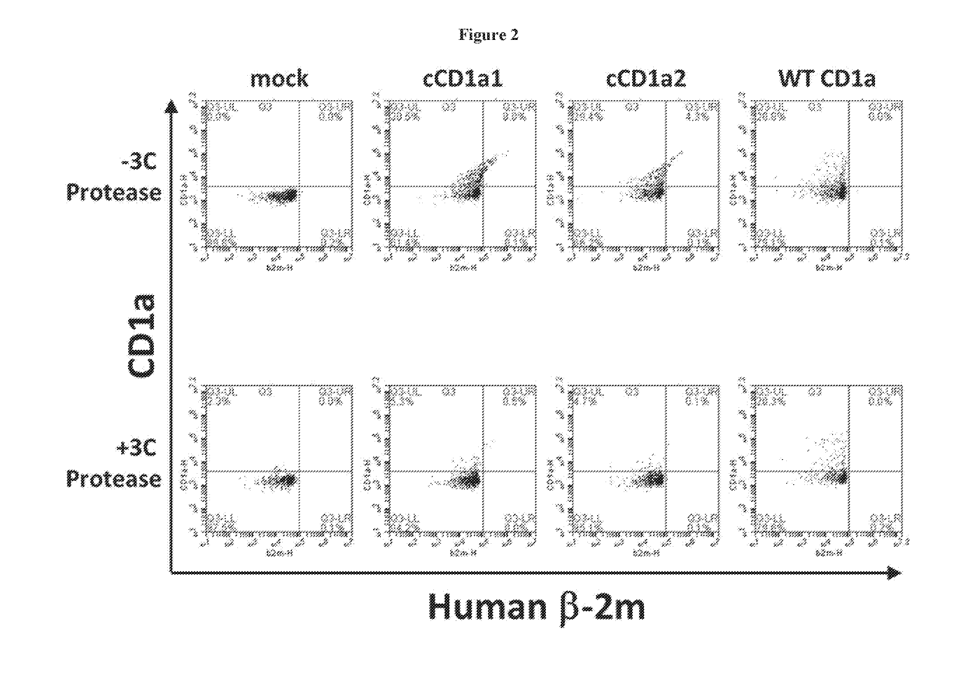

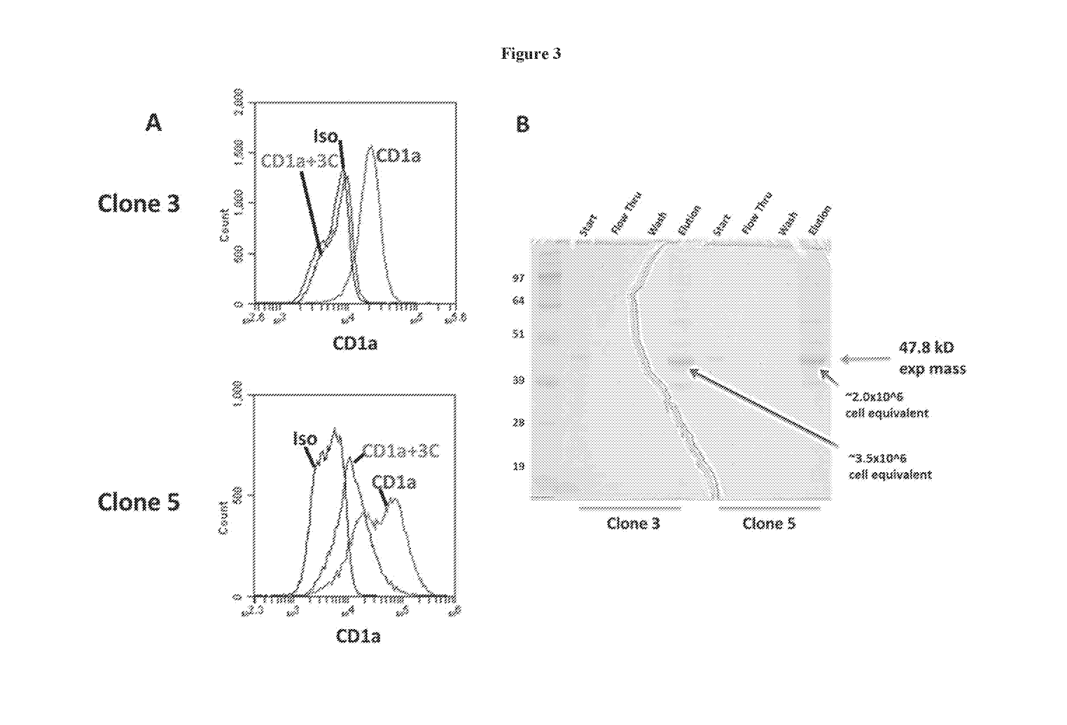

To verify that the newly engineered cleavable CD1a (cCD1a) gene could be expressed on the cell surface, mouse B16-F10 melanoma cells (which lack both human CD and β-2m) were transiently transfected with two different preparations of the cCD1a construct or wild-type CD1a and surface stained with antibodies specific for CD1a and human β-2m with or without 3C protease treatment (FIG. 2). A decrease in cell surface CD1a and human β-2 microglobulin expression is only observed in the cells expressing cCD1a and not wtCD1a, demonstrating the specific nature of the protease-based cleavage of cCD1a but not wtCD1a from the cell surface. To create a system yielding sufficient cCD1a expression for downstream preparative biochemical purification, the cCD1a transgene was sub-cloned into a plasmid allowing mammalian drug selection to enable the generation of two clones stably producing cCD1a in the human HeLa cell line background, and the product of the transgene was again shown to be sensitive to 3C protease cleavage (FIG. 3A). SDS-PAGE evaluation of the cCD1a protein demonstrates a band corresponding to the expected size in both the starting material and the final eluate (FIG. 3B).

### EXAMPLE 3

### Antigen Isolation

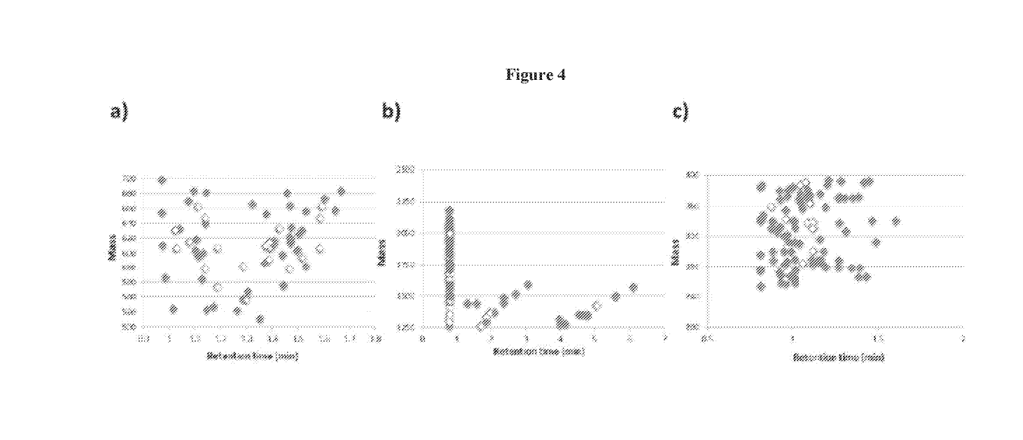

To evaluate the antigen binding capacity of the cCD1a protein, cells derived from the higher expressing stable clone 5 were incubated with M. tuberculosis lysate, and the cCD1a protein was cleaved and purified, with cCD1a collected from untreated cells serving as a negative control. Mass spectrometric evaluation of fractions collected from Ni-NTA-bound cCD1a proteins generated from this experiment using increasingly nonpolar/hydrophobic solvents revealed the presence of a spectrum of mycobacterial lipid antigens unique to the antigen-treated group but not observed in the control sample (FIG. 4). These findings provide “proof-of-concept” data confirming the ability of the cCD1a system as a means of querying a wider milieu of antigens than existing strategies and highlight the flexibility of the 3C protease system as a means of preparing proteins displayed on mammalian cell surfaces.

### EXAMPLE 4

### cCD1a Function is Preserved

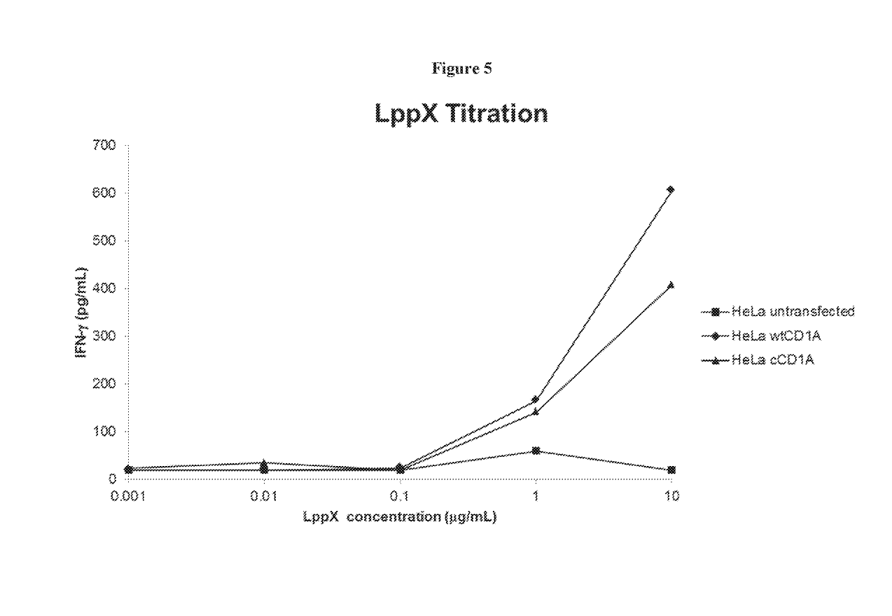

HeLa cells were transfected with wild-type CD (wtCD1A) or a construct comprising a CD1a extracellular domain and an engineered protease cleavage site between the extracellular domain and a transmembrane domain (cCD1A). 20,000 transfected HeLa cells were incubated with 10,000 LCD4.G T cells and 0.001-10 μg/mL LppX in a 96 well plate for each of wtCD1A transfected cells and cCD1A cells. LCD4.G is a CD restricted T cell clone capable of recognizing a lipoprotein LppX antigen. The cells were incubated in RPMI media comprising 10% human serum. Untransfected HeLa cells incubated with LCD4.G cells and LppX were used as a negative control. Interferon γ (INF-γ) production was monitored by ELISA. HeLa cells transfected with cCD1A were capable of inducing INF-γ production to a similar extent as HeLa cells transfected with wtCD1a, and increasing concentrations of lipoprotein LppX induced increasing concentrations of INF-γ (FIG. 5). The negative control did not induce any appreciable amount of INF-γ. These results suggest that cCD1A is capable of presenting antigens to T cells.

### INCORPORATION BY REFERENCE

All of the U.S. patents, U.S. published patent applications, foreign patents, foreign patent publications, and other publications cited herein are hereby incorporated by reference.

### EQUIVALENTS

Those skilled in the art will recognize, or be able to ascertain using no more than routine experimentation, many equivalents to the specific embodiments of the invention described herein. Such equivalents may have the following characteristics.

1. A protein comprising:
an extracellular domain comprising the extracellular domain of an antigen-presenting protein;
a transmembrane domain; and
a protease cleavage site located between the extracellular domain and the transmembrane domain;
wherein:
the protease cleavage site is located in proximity to the transmembrane domain; and
the protease that recognizes the protease cleavage site recognizes an amino acid sequence comprising at least 4 amino acids.

2. The protein of claim 1, wherein the antigen-presenting protein is a CD1 protein.

3. The protein of claim 2, wherein the antigen-presenting protein is CD1a, C1b, CD1c, CD1d, or CD1e.

4. A protein comprising:
an extracellular domain;
a transmembrane domain; and
a protease cleavage site located between the extracellular domain and the transmembrane domain;
wherein:
the protease cleavage site is located in proximity to the transmembrane domain; and
the protease that recognizes the protease cleavage site recognizes an amino acid sequence comprising at least 4 amino acids.

5. The protein of claim 1, wherein the transmembrane domain consists of a single alpha helix.

6. The protein of claim 1, wherein the protease cleavage site comprises a recognition sequence for thrombin, factor Xa, TEV protease, enteropeptidase, or rhinovirus 3C protease.

7. (canceled)

8. The protein of claim 1, wherein the protease cleavage site comprises the amino acid sequence set forth in any one of SEQ ID NO:1, SEQ ID NO:2, SEQ ID NO:3, SEQ ID NO:4, or SEQ ID NO:5.

9. (canceled)

10. The protein of claim 1, further comprising a first affinity tag located between the N-terminus of the protein and the protease cleavage site.

11. The protein of claim 10, wherein the first affinity tag is located between the extracellular domain and the protease cleavage site.

12. The protein of claim 10, further comprising a second affinity tag located between the C-terminus of the protein and the protease cleavage site.

13. The protein of claim 12, wherein the second affinity tag is located between the transmembrane domain and the protease cleavage site.

14. (canceled)

15. The protein of claim 13, wherein at least one of the first affinity tag and the second affinity tag comprises an amino acid sequence set forth in any one of SEQ ID NO:6-24.

16. The protein of claim 13, further comprising a N-terminal leader sequence for translocating the extracellular domain across a membrane.

17. The protein of claim 16, further comprising a cytosolic domain for trafficking the protein within a cell.

18. The protein of claim 17, wherein the cytosolic domain comprises the cytosolic domain of CD1a.

19. A cell comprising a gene encoding the protein of claim 17.

20-40. (canceled)

41. A method for isolating an antigen, comprising:
contacting a cell of claim 19 with a mixture comprising the antigen;
incubating the cell with a protease that recognizes the protease cleavage sequence, thereby cleaving the extracellular domain from the cell; and
isolating the extracellular domain from the mixture, thereby isolating the antigen.

42. A method for isolating an antigen, comprising:
contacting a cell of claim 19 with a mixture comprising the antigen;
isolating the cell from the mixture;
incubating the cell with a protease that recognizes the protease cleavage sequence, thereby cleaving the extracellular domain from the cell; and
isolating the extracellular domain from the cell, thereby isolating the antigen.

43. (canceled)

44. A method for isolating a T cell, comprising:
contacting a cell of claim 19 with an antigen;
incubating the cell with a plurality of T cells;
isolating a T cell that binds to the cell, thereby isolating the T cell; and
incubating the cell with a protease that recognizes the protease cleavage sequence, thereby cleaving the T cell from the cell.

45-49. (canceled)

50. A nucleic acid encoding the protein of claim 17.
